(19) United States
(12) Patent Application Publication
(10) Pub. No.: US 2021/0023336 A1
(43) Pub. Date: Jan. 28, 2021

(54) Vascular Access Apparatus

(71) Applicant: Choon Kee Lee, Denver, CO (US)

(72) Inventor: Choon Kee Lee, Denver, CO (US)

(21) Appl. No.: 16/524,127

(22) Filed: Jul. 28, 2019

(51) Int. Cl. A61M 25/00; A61M 25/06; A61M 1/36

(52) U.S. Cl. CPC A61M 25/0028; A61M 25/0606; A61M 2025/0031; A61M 1/3653; A61M 2025/0039; A61M 25/0637

(57) ABSTRACT

The present invention for vascular access comprises a single-lumen catheter that is configured to be reversibly and coaxially combinable with an inner catheter so as to form a coaxial double-lumen catheter device. The single-lumen catheter in a configuration of the coaxial double-lumen catheter device is configured to take in blood from patient to a hemodialysis machine whereas the inner catheter in the configuration of the coaxial double-lumen catheter device is configured to return the blood from the hemodialysis machine to the patient.

### CROSS REFERENCE TO RELATED APPLICATIONS

Attached please refer to the Information Disclosure Statement for the cross reference to related applications.

### TECHNICAL FIELD

The present invention relates generally to the field of vascular access devices. More specifically, the present invention provides a vascular access for hemodialysis.

### BACKGROUND OF THE INVENTION

Consistent and dependable access to blood vessels is one of most important technical requirements for successful long-term hemodialysis for patients in renal failure requiring therapy with dialysis. Currently there are two main approaches to secure an access to the blood vessels, one with a temporary catheterization of internal jugular, subclavian or femoral veins with a central venous catheter and the other with an arteriovenous fistula mostly on forearm blood vessels.

The arteriovenous fistula has been shown to be associated with significantly less incidence of infectious and mechanical complications than the temporary catheterization using the central venous catheter, making it an access of choice for the long-term hemodialysis. Yet, the arteriovenous fistula is known to have a range of complications, from non-mechanical to mechanical complications. The non-mechanical complications comprise high output congestive heart failure, venous congestion, arterial steal syndrome, lymphedema, and infection. The mechanical complications include thrombosis, venous stenosis, aneurysm/pseudoaneurysm, hematoma of a puncture site, and unstoppable bleeding and erosion/ulceration of a puncture site.

Except for the thrombosis and the venous stenosis, the majority of the mechanical complications are directly or indirectly related to needle puncture of a vein connected to the arteriovenous fistula. Each hemodialysis session requires two puncture sites on the vein connected to the arteriovenous fistula, including the first puncture site for a single-lumen needle/catheter for blood intake and the second puncture site for another single-lumen needle/catheter for blood output. Over a lifetime use of the arteriovenous fistula, the incidence of the mechanical complications of a particular patient can be reduced by half if number of the puncture sites is reduced from two to one per one session of the hemodialysis. A double-lumen catheter device for puncturing the vein of the arteriovenous fistula can be used to achieve a goal of reducing the number of the puncture site to one per each session of the hemodialysis. The first lumen of the double-lumen catheter is configured for taking in blood, and the second lumen of the double-lumen catheter for returning the blood.

There are a few considerations for the double-lumen catheter device for accessing the vein connected to the arteriovenous fistula: 1) Size (Gauge) of the double-lumen catheter device should be minimally larger than a single-lumen needle/catheter for accessing the arteriovenous fistula in order to minimize a size of a puncture wound on a venous wall of the vein connected to the arteriovenous fistula; 2) Insertion into and removal of the double-lumen catheter from the vein should minimize trauma to the venous wall as much as possible; 3) A blood intake catheter of the double-lumen catheter should have an adequate blood intake area to bring in a sufficient amount of blood for optimal hemodialysis; 4) The blood intake catheter should maintain an original tubular configuration of the blood intake catheter during hemodialysis without cross-sectional collapse of the tubular configuration so as to assure continuous delivery of blood for hemodialysis; 5) A blood output catheter should have a tip of the blood output catheter separated from the blood intake area of the blood intake catheter by a sufficient distance in order to avoid blood recirculation between the blood intake catheter and the blood output catheter; 6) Blood ejecting from the tip of the blood output catheter should be directed toward the heart.

### SUMMARY OF THE INVENTION

In one embodiment, the present invention comprises an outer single-lumen catheter and an inner single-lumen catheter in a longitudinally ridged tubular configuration which is configured to be reversibly and coaxially threaded in a tubular space of the outer single-lumen catheter so as to reversibly form a coaxial double-lumen catheter device. The outer single-lumen catheter is configured to be interchangeably used either as a stand-alone catheter or as an outer catheter of the coaxial double-lumen catheter. In the configuration of the coaxial double-lumen catheter, the outer single-lumen catheter is configured to serve as a blood intake catheter, and the inner single-lumen catheter is configured to serve as a blood output catheter. The outer single-lumen catheter is configured to fit slidably over a tubular needle which is to penetrably puncture a blood vessel of an arteriovenous fistula for hemodialysis.

In one embodiment, the outer single-lumen catheter provided in a tubular configuration comprises a proximal portion having a proximal opening, and a distal portion having a sealable lock assembly. The proximal opening is configured to take in blood to the tubular space of the outer single-lumen catheter. The sealable lock assembly comprises an external helical thread portion and a seal portion, arranged in tandem, and is configured to sealably accommodate and lock in the inner single-lumen catheter. A mid portion of the outer single-lumen catheter comprises a tubular shaft which connects the proximal portion to the distal portion. A sidewall of the tubular shaft of the mid portion is connected in a Y configuration to an outgoing tube having a connecting hub and an attachment base plate. The outgoing tube is configured to adjoin the sidewall and open to a tubular space of the tubular shaft.

In one embodiment, the proximal opening of the outer single-lumen catheter insertable in the blood vessel directed toward the heart is configured to suck in a high-speed arterial blood delivered from an artery of the arteriovenous fistula in a direction away from the heart. A blood flow at a flow rate of F₁ heading toward a hemodialysis machine from the proximal opening where the blood is taken in by a negative pressure generated by the hemodialysis machine is in an opposite direction to the direction of an arterial blood flow at a flow rate of F₂ toward the heart. At an edge of the proximal opening serving as a velocity boundary, the arterial blood is sucked in across the edge at a transitional flow rate of F₃>F₁+F₂. In this modeling of the blood flow across the proximal opening of the outer single-lumen catheter, the blood flow goes from the F₁ to the F₃ to the F₂, wherein the F₁ is opposite to the F₂. Since the F₃ is faster than either the F₁ or the F₂, a flow pressure P₃ of the F₃ at the edge of the proximal opening is lower than a flow pressure P₁ of the F₁ in the mid portion of the outer single-lumen catheter or a flow pressure P₂ of the F₂ in the artery of the arteriovenous fistula. Consequently, a diameter of the edge of the proximal opening can be dynamically varied, always getting narrowed, proportionally by a degree of a decrease in the flow pressure P₃. In short there is a risk of cross-sectional collapse of the edge as the velocity boundary of the proximal opening. One method to avoid the issue is to widen a surface area of the velocity boundary by changing a configuration of a circle of the velocity boundary to an elliptical configuration. An opening area (surface area: S) of an elliptical configuration of the proximal opening is calculated in an equation of π×r₁×r₂, wherein the r₁ is a long radius and the r₂ is a short radius of the elliptical proximal opening, and wherein the r₂ is the same as a radius of tubular shaft of the mid portion. Since the S of an ellipse increases proportionally to the r₁, and the Hagen-Poiseuille equation states that flow rate is proportional to the S², an increase in the r1 potentially reduces a drop in the flow pressure P₃, and thereby, the cross-sectional collapse of the proximal opening. Thus, the proximal opening of the prevent invention is provided in a beveled, elliptical configuration having the r₁>2×r₂ so as to reduce/avoid the cross-sectional collapse of the proximal opening of the outer single-lumen catheter by changes in the flow pressure P₃ of the F₃.

In one embodiment, the present invention aims to reduce turbulence of blood flow across the proximal opening of the outer single-lumen catheter. A 180° change in the direction of the blood flow across the velocity boundary of the proximal opening, i.e., the F₁ is opposite to the F₂., generates the turbulence of the blood flow and consequent shear stress on the outer single-lumen catheter. The turbulence of the blood flow impacts on performance of the outer single-lumen catheter, and the shear stress to the outer single-lumen catheter increases a rate of wear of the outer single-lumen catheter. The turbulence is dependent on Reynolds number of a particular fluid in a pipe system, wherein the Reynolds number is proportional to a circular diameter of the pipe but inversely proportional to a cross-sectional area of the pipe: Re=QD_{H}/vA (Re—Reynolds number; Q—flow rate; D_{H}—hydraulic diameter of the pipe; v—viscosity of the fluid; A—pipe's cross-sectional area). Since the D_{H} of the proximal opening in the beveled, elliptical configuration remains the same as the diameter of the tubular shaft of the outer single-lumen catheter, and a viscosity of a blood of a patient remains constant, the Reynolds number expected for the proximal opening depends on the flow rate and the cross-sectional area of the proximal opening. Essentially, the larger the cross-sectional area of the velocity boundary of the proximal opening, the lower the Reynolds number becomes, translating into a lesser degree of the turbulence. The proximal opening in the beveled, elliptical configuration having the r₁>2×r₂ is configured to increase the cross-sectional area of the velocity boundary, thereby reducing the Reynolds number of the blood flowing through the outer single-lumen catheter, wherein a lower Reynolds number translates to the lesser degree of the turbulence of the blood and a lesser degree of the shear stress in and around the outer single-lumen catheter.

In one embodiment, the sealable lock assembly disposed thereof at the distal portion of the outer single-lumen catheter comprises the external helical thread portion which is provided in a cylindrically tubular configuration. The external helical thread portion comprises an external helical thread disposed on an outer surface of a cylindrical tube of the external helical thread portion, and a plurality of longitudinal grooves disposed on an inner surface of the cylindrical tube along a longitudinal axis of the cylindrical tube. The external helical thread is configured to be helically threaded over by a rotatable fastener having a matching internal helical thread to the external helical thread. The rotatable fastener is a part of the inner single-lumen catheter. Each longitudinal groove of the cylindrical tube is configured to reversibly and leakproofly mate with each corresponding rectangularly protruding tongue of a male key portion of the inner single-lumen catheter.

In one embodiment, the seal portion of the sealable lock assembly comprises a cylindrical seal chamber and a two-part elastomeric seal sealably encased by the cylindrical seal chamber. The seal portion is located proximal to and distally adjoins the external helical thread portion. The two-part elastomeric seal is made of a polymeric elastomer such as silicone to impart rubbery elasticity and compressibility. The two-part elastomeric seal is provided as a solid elastomer in a cylindrical configuration. The two-part elastomeric seal comprises a distal part proximally adjoining a proximal part. The distal part of the two-part elastomeric seal is provided as a cylindrical tube and non-dilatable, and comprises a plurality of linear grooves on an inner surface of the distal part in the cylindrical tubular configuration. The plurality of the linear grooves of the distal part are configured to leakproofly mate with a plurality of linear ridges of the inner single-lumen catheter in the longitudinally ridged tubular configuration during insertion of the inner single-lumen catheter into the outer single-lumen catheter, thus preventing spillage of blood from the distal part of the outer single-lumen catheter.

In one embodiment, the proximal part of the elastomeric seal is provided in a solid elastomeric cylinder, and comprises a dilatable linear slit provided centrally in the solid elastomeric cylinder along a longitudinal axis of the solid elastomeric cylinder. The dilatable linear slit is to be closed shut and to seal off the seal portion in an unengaged configuration, and to be dilatably open to accommodate the tubular shaft portion of the inner single-lumen catheter penetrably disposed inside the dilatable linear slit in an engaged configuration with said tubular shaft portion of the inner single-lumen catheter. Separately, the dilatable linear slit is to be closed shut and to seal off the seal portion in an unengaged configuration, and to be dilatably open to sealably accommodate a tubular shaft portion of the tubular needle penetrably disposed inside the dilatable linear slit in an engaged configuration with the tubular shaft portion of the tubular needle. The outer single-lumen catheter is configured to be assembled with the tubular needle reversibly inserted inside the outer single-lumen catheter for penetrably puncturing the blood vessel of the arteriovenous fistula. Once the outer single-lumen catheter assembled with the tubular needle is securely in place inside the blood vessel, the tubular needle is withdrawn from the outer single-lumen catheter. The dilatable linear slit of the two-part elastomeric seal is closed shut following the withdrawal of the tubular needle, thus preventing spillage of the blood from the distal portion of the outer single-lumen catheter.

In one embodiment, the elastomeric seal is configured for a two-step sequential sealing of said elastomeric seal when engaging with the inner single-lumen catheter. The dilatable linear slit is configured with a distal opening distally facing the cylindrical tube of the distal part, and a proximal opening proximally facing a tubular lumen of the outer single-lumen catheter. A proximal portion of the inner single-lumen catheter is configured in a way that a distance between a proximal opening of the inner single-lumen catheter and a proximal end of the plurality of the longitudinal ridges of the inner single-lumen catheter is shorter than a longitudinal length of the cylindrical tube of the distal part of the elastomeric seal. The first step of sealing of the elastomeric seal occurs at the proximal opening of the elastomeric seal when the proximal opening of the inner single-lumen catheter is introduced into the distal opening of the dilatable linear slit. The proximal opening of the dilatable linear slit stays closed shut at the time the distal opening of the dilatable linear slit is open by the proximal opening of the inner single-lumen catheter. The second step of the sealing occurs when the proximal end of the plurality of the longitudinal ridges of the inner single-lumen catheter begins to get leakproofly engaged with the plurality of the linear grooves of the distal part of the elastomeric seal. Once the plurality of the linear grooves of the distal part are fully and leakproofly engaged with the plurality of the longitudinal ridges of the inner single-lumen catheter, the proximal opening of the dilatable linear slit opens and lets the inner single-lumen catheter proximally advance. This two-step sealing by the two-part elastomeric seal is to assure of prevention of blood spillage during an introduction of the inner single-lumen catheter into the outer single-lumen catheter.

In one embodiment, the two-part elastomeric seal is provided in a configuration of a corrugated roll with a plurality of longitudinal ridges and furrows disposed on an outer circumferential surface of the two-part elastomeric seal along a longitudinal axis of the two-part elastomeric seal. The longitudinal ridges are configured to be compressible against a cylindrical housing of the cylindrical seal chamber when the tubular shaft portion of the inner single-lumen catheter is penetrably introduced inside the dilatable linear slit of the two-part elastomeric seal. In the unengaged configuration, the longitudinal ridges protrude from the outer circumferential surface of the two-part elastomeric seal, filling up and sealing the cylindrical housing. In the engaged configuration with the tubular shaft portion of the inner single-lumen catheter introduced inside the dilatable linear slit of the two-part elastomeric seal, the longitudinal ridges are radially compressed against the cylindrical housing so as to let the dilatable linear slit open and accommodate the tubular shaft portion of the inner single-lumen catheter in a tubular space reversibly made in the dilatable linear slit. Radial compression of a longitudinal ridge results in a loss of a vertical height of the longitudinal ridge and a loss of a cylindrical volume of the two-part elastomeric seal. The loss of a cylindrical volume (Vₛ) of the two-part elastomeric seal by the radial compression of the plurality of the longitudinal ridges from an uncompressed configuration to a compressed configuration of the two-part elastomeric seal is calculated as Vₛ=Vₐ-V_{b}; Vₐ=h×π×rₐ²; V_{b}=h×π×r_{b}², wherein Vₐ is a cylindrical volume of the two-part elastomeric seal in the uncompressed configuration; rₐ is a radius of the two-part elastomeric seal from an axial center to an outer periphery of the longitudinal ridge in the uncompressed configuration; r_{b} is a radius of the two-part elastomeric seal from the axial center to an outer periphery of the longitudinal ridge in the compressed configuration; h is a height of the two-part elastomeric seal. Vₛ is configured to be equivalent to a cylindrical volume (V_{d}=h×π×r_{d}²) of the dilatable linear slit in a dilated configuration having the inner single-lumen catheter introduced inside the dilatable linear slit, wherein r_{d} is a radius of the dilatable linear slit in the dilated configuration. Hence, (h×π×rₐ²)−(h×π×r_{b}²)=h×π×r_{d}²; r_{d}=√(rₐ²-r_{b}²). Therefore, a radius of the dilatable linear slit in a dilated configuration fully engaged with the inner single-lumen catheter is configured to be equal to or less than a square root of a sum of (the radius² of the two-part elastomeric seal from an axial center to an outer periphery of the longitudinal ridge in the uncompressed configuration—the radius² of the two-part elastomeric seal in the compressed configuration): r_{d}≤√(rₐ²-r_{b}²).

In one embodiment, the mid portion of the outer single-lumen catheter comprises an outer tube fixedly encircling a portion of the tubular shaft of said outer single-lumen catheter in a tube-in-tube configuration. The outgoing tube of the mid portion adjoins at an angle the outer tube of the outer single-lumen catheter and opens to a sidewall of the outer tube and the sidewall of tubular shaft. The outgoing tube is configured to serve as conduit for an inflow of blood flowing from the proximal opening of the outer single-lumen catheter to a hemodialysis machine. A distal portion of the outgoing tube is connected to a connecting hub which is configured to get reversibly connected to an extension tubing. The attachment base plate of the mid portion is fixedly connected to a lower part of the outer tube of the mid portion, and is configured to serve for fastening the arteriovenous fistula access device to an underlying skin.

In one embodiment, the inner single-lumen catheter provided in the longitudinally ridged tubular configuration comprises a proximal portion having a proximal opening in a circular configuration, a distal portion having a sealable key assembly, a connecting tube, and the rotatable fastener slidably placed over the connecting tube, and a mid portion having a tubular shaft. The mid portion connects the proximal portion to the distal portion. The proximal portion of the inner single-lumen catheter is configured to protrude for a distance from the proximal opening of the outer single-lumen catheter so as to avoid recirculation of the blood between the proximal opening of the outer single-lumen catheter and the proximal opening of the inner single-lumen catheter. The inner single-lumen catheter comprises a plurality of longitudinal ridges arising from an outer circumferential surface of the proximal and mid portions of the inner single-lumen catheter. The longitudinal ridges of the inner single-lumen catheter are configured to separate an inner circumferential surface of the outer single-lumen catheter from the outer circumferential surface of the inner single-lumen catheter at least by a vertical height of a longitudinal ridge. In between the inner circumferential surface of the outer single-lumen catheter and the outer circumferential surface of the inner single-lumen catheter, there is provided a longitudinal tubular space in a doughnut configuration on radial cross-section. The longitudinal tubular space serves as a conduit for the inflow of the blood whereas the inner single-lumen catheter serves a conduit for an outflow of the blood.

In one embodiment, the sealable key assembly of the inner single-lumen catheter is provided as a cylindrical tube proximally connected to the tubular shaft of the mid portion of the inner single-lumen catheter. The cylindrical tube of the sealable key assembly distally adjoins the connecting tube of the inner single-lumen catheter. The sealable key assembly comprises the male key portion having a plurality of rectangularly protruding tongues circumferentially disposed thereof at a proximal portion of the cylindrical tube of the sealable key assembly in a symmetrical configuration across the proximal portion of the cylindrical tube of the sealable key assembly. A rectangularly protruding tongue of the inner single-lumen catheter is made of a hard polymer, and is configured to slidably and sealably mate with a corresponding longitudinal groove of the sealable lock assembly of the outer single-lumen catheter. In a mid portion of the cylindrical tube of the sealable key assembly, a circular flange protrudes from an outer surface of the cylindrical tube of the sealable key assembly. The circular flange is configured to mate with the rotatable fastener of the inner single-lumen catheter in a way the circular flange provides a leakproof seal inside the rotatable fastener when the rotatable fastener fully fastens the cylindrical tube of the sealable key assembly of the inner single-lumen catheter to the sealable lock assembly of the outer single-lumen catheter.

In one embodiment, the sealable key assembly comprises a couple of planar leaves fixedly adjoining at an angle a distal end of the cylindrical tube of the sealable key assembly. A planar leaf, provided as a thermoplastic rectangular plate, is configured with one edge of said planar leaf adjoining a portion of a circumference of a distal end of the cylindrical tube of the sealable key assembly. The planar leaves are configured to distally protrude out from and abut a distal end of the rotatable fastener when the rotatable fastener fully fastens the cylindrical tube of the sealable key assembly of the inner single-lumen catheter to the sealable lock assembly of the outer single-lumen catheter. The planar leaves abutting the distal end of the rotatable fastener is configured to prevent backward slippage of the inner single-lumen catheter from the outer single-lumen catheter once the inner single-lumen catheter is fully assembled with the outer single-lumen catheter.

In one embodiment, the vascular access apparatus of the present invention can be used for accessing other blood vessels where a double lumen catheter is required. In many instances, a vascular catheter is used for administering several drugs simultaneously through said vascular catheter. Problems may occur when some of the drugs are not compatible with each other for the simultaneous administration. In other instances, especially under emergent situations, a time required for an introduction of a catheter through an introducer by laborious steps of Seldinger technique may be reduced by the present invention which obviates use and removal of the introducer. The outer single-lumen catheter of the present invention itself is configured as one device for a needle to puncture a vessel, an introducer that dilates a needle track and the vessel and does not need to be withdrawn, and an outer catheter.

### BRIEF DESCRIPTION OF THE DRAWINGS

FIG. 1 shows a schematic presentation of an arteriovenous fistula access device having an inner single-lumen catheter insertably assembled with an outer single-lumen catheter.

FIGS. 2A˜2C represent schematic illustrations of the outer single-lumen catheter; FIGS. 2D˜2E show a schematic view of a tubular needle and the outer single-lumen catheter assembled with the tubular needle, respectively.

FIG. 3A depicts a schematic three-dimensional cut-out view of a portion of the outer single-lumen catheter; FIG. 3B illustrates a schematic example of a two-part elastomeric seal, showing an distal opening; FIG. 3C illustrates a schematic example of the two-part elastomeric seal, showing a proximal opening; FIG. 3D shows a two-dimensional view of the two-part elastomeric seal; FIG. 3E shows an in-situ placement of the two-part elastomeric seal inside a cylindrical seal chamber of a sealable lock assembly of the outer single-lumen catheter.

FIGS. 4A˜4F show a schematic example of components of the inner single-lumen catheter.

FIGS. 5A˜5B show schematic illustrations of an assembly of the sealable lock assembly of the outer single-lumen catheter with a sealable key assembly of the inner single-lumen catheter.

FIGS. 6A˜6B show schematic three-dimensional cut-out views of the two-part elastomeric seal encased in the cylindrical seal chamber, in an assembly with the tubular needle inserted inside the outer single-lumen catheter; FIGS. 6C˜6D show schematic cross-sectional views of a proximal portion of the two-part elastomeric seal, without and with the tubular needle inserted inside the outer single-lumen catheter.

FIGS. 7A˜7B show schematic three-dimensional cut-out views of the two-part elastomeric seal encased in the cylindrical seal chamber, in an assembly with the inner single-lumen catheter inserted inside the outer single-lumen catheter; FIGS. 7C˜7D show schematic cross-sectional views of a distal portion of the two-part elastomeric seal, without and with the inner single-lumen catheter inserted inside the outer single-lumen catheter.

FIGS. 8A˜8D show a schematic three-dimensional cut-out view and schematic cross-sectional views of an example of the arteriovenous fistula access device inserted inside a blood vessel.

FIGS. 9A˜9E show schematic three-dimensional cut-out views and schematic cross-sectional views of an example of a mating configuration of a portion of the sealable lock assembly with a distal portion of the tubular needle and with the sealable key assembly of the inner single-lumen catheter, respectively.

FIGS. 10A˜10E show schematic views of an example of a rotatable fastener of the inner single-lumen catheter fastening the sealable key assembly of the inner single-lumen catheter to the sealable lock assembly of the outer single-lumen catheter.

FIGS. 11A˜11G illustrate a schematic example of a two-step sealing of an elastomeric seal of the outer single-lumen catheter.

### DETAILED DESCRIPTION OF THE DRAWINGS

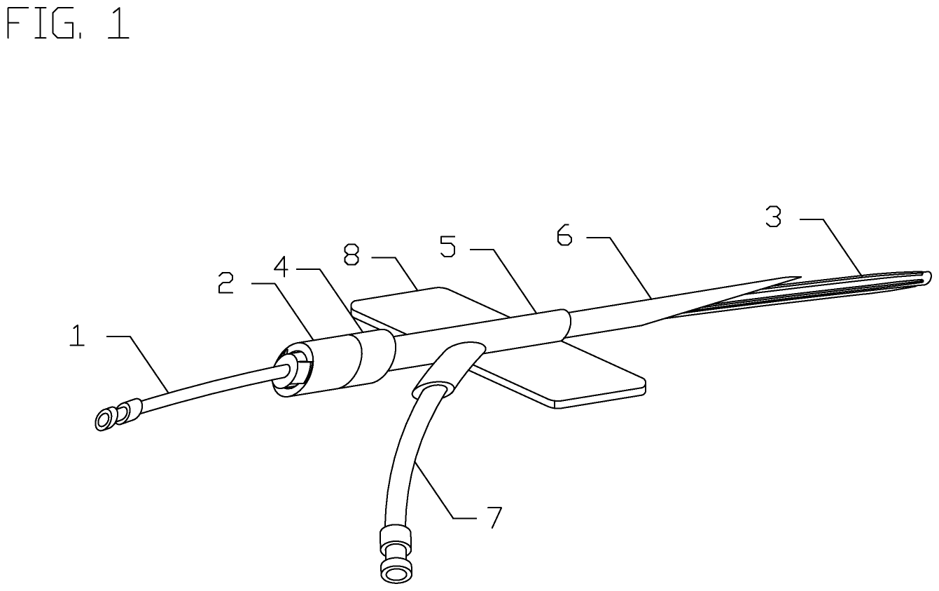

As described below, the present invention provides an arteriovenous fistula access device comprising an outer single-lumen catheter and an inner single-lumen catheter. It is to be understood that the descriptions are solely for the purposes of illustrating the present invention, and should not be understood in any way as restrictive or limited. Embodiments of the present invention are preferably depicted with reference to FIGS. 1 to 11, however, such reference is not intended to limit the present invention in any manner. The drawings do not represent actual dimension of devices, but illustrate the principles of the present invention.

FIG. 1 shows a schematic presentation of an arteriovenous fistula access device having an inner single-lumen catheter comprising a proximal portion 3 in a tubular configuration and a rotatable fastener 2 and a connecting tube 1 of a distal portion. The inner single-lumen catheter is reversibly assembled with an outer single-lumen catheter which comprises a proximal portion 6 in a tubular configuration, an outer overtube 5, an attachment base plate 8 and an outgoing tube 7 of a mid portion, and a cylindrical seal chamber 4 of a sealable lock assembly of a distal portion. The proximal portion 3 of the inner single-lumen catheter protrudes from a proximal opening of the proximal portion 6 of the outer single-lumen catheter for a distance. Both the proximal portions 3 and 6 are configured to be slidably inserted in a blood vessel connected to an arteriovenous fistula. The rotatable fastener 2 tightly fastens the inner single-lumen catheter toward a distal end of the cylindrical seal chamber 4 of the sealable lock assembly. The connecting tube 1 of the inner single-lumen catheter is configured to serve as a conduit for an outflow of blood, whereas the outgoing tube 7 of the outer single-lumen catheter is configured to serve as a conduit for an inflow of the blood. The attachment base plate 8 is configured to serve as an anchoring site for the outer single-lumen catheter to an underlying skin of patient.

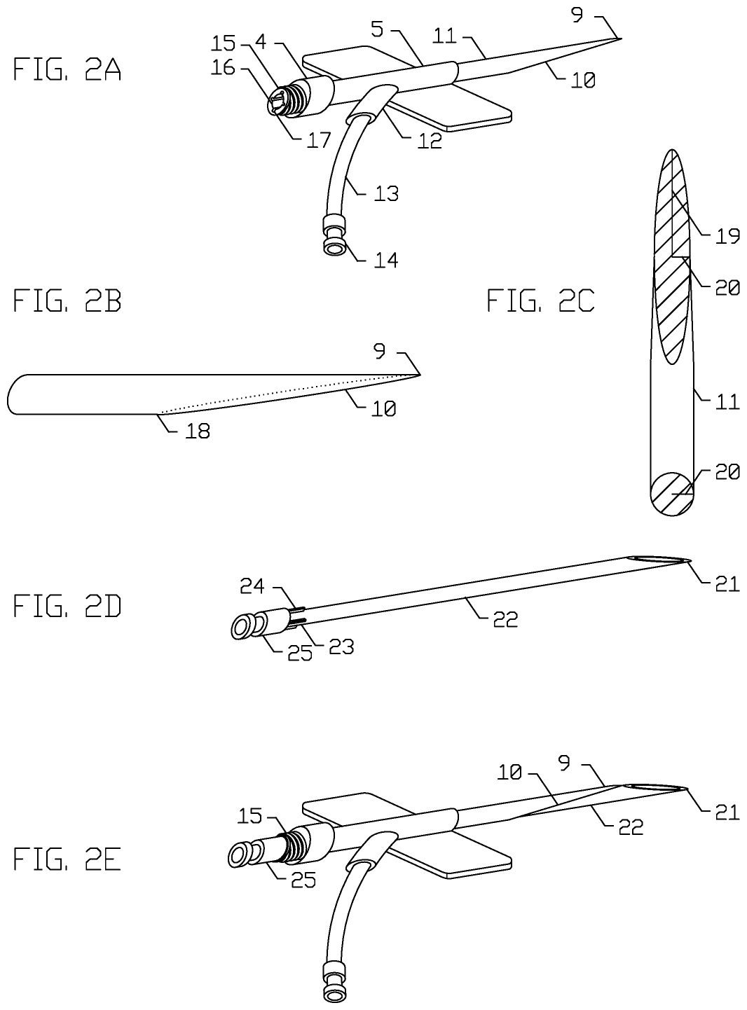

FIG. 2A shows a schematic illustration of the outer single-lumen catheter in an unassembled configuration without the inner single-lumen catheter. The proximal portion comprises a portion of a tubular shaft 11, an elliptical proximal opening 10 in a beveled configuration and a proximal tip 9. The tubular shaft 11 at the mid portion is encircled by the outer overtube 5 in a tube-in-tube configuration. Referring to the outgoing tube 7 of FIG. 1, the outgoing tube 7 comprises a connecting tubular flange 12 adjoining a sidewall of the outer overtube 5 and opening into the tubular shaft 11, a connecting tube 13 and a connecting hub 14. The sealable lock assembly of the distal portion comprises the cylindrical seal chamber 4 distally adjoining an external helical thread portion 15 in a cylindrical tubular configuration. The external helical thread portion 15 comprises a cylindrical tube 16 having a plurality of longitudinal grooves 17 disposed on an inner surface of the cylindrical tube 16 along a longitudinal axis of the cylindrical tube.

FIG. 2B illustrates an schematic example of the beveled configuration of the elliptical proximal opening 10 obliquely placed in between the proximal tip 9 and a recessed point 18 of the tubular shaft on a two dimensional lateral view. In a two dimensional bottom-up view of FIG. 2C, the elliptical proximal opening 10 is shown in an elliptical configuration having a long radius (r₁) 19 and a short radius (r₂) 20. The short radius 20 is identical to a perpendicularly measured radius of the tubular shaft 11. The long radius 19 of the present invention is configured to be longer than 2×r₂ (the short radius 20): r₁>2×r₂. Consequently, an elliptical area (S₁) of the elliptical proximal opening 10 is larger by a cross-sectional circular area (S₂) of the tubular shaft 11 than the cross-sectional circular area of the tubular shaft 11: S₁-S₂=π×(>2×r₂²-r₂²).

FIG. 2D shows a schematic view of the tubular needle comprising a needle tip 20, a tubular shaft portion 22, a distal portion 23 having a plurality of rectangularly protruding tongues 24 circumferentially disposed thereof at the distal portion of the tubular needle and connecting hub 25. In FIG. 2E, the outer single-lumen catheter is shown as assembled with the tubular needle: The needle tip 20 and a proximal portion of the tubular shaft portion 22 of the tubular needle protrude from the elliptical proximal opening 10 and the proximal tip 9 of the outer single-lumen catheter. The elliptical proximal opening 10 of the outer single-lumen catheter is configured to tightly encircle the tubular shaft portion 22 of the tubular needle. The connecting hub 25 of the distal portion of the tubular needle tightly abuts the external helical thread portion 15 of the outer single-lumen catheter, so as to fully insert the rectangularly protruding tongues 24 of FIG. 2D into the longitudinal grooves 17 of the cylindrical tube 16 of FIG. 2A. The full insertion of the rectangularly protruding tongues 24 of FIG. 2D into the longitudinal grooves 17 of the cylindrical tube 16 of FIG. 2A is configured to maintain a reversible leakproof seal of the cylindrical tube 16.

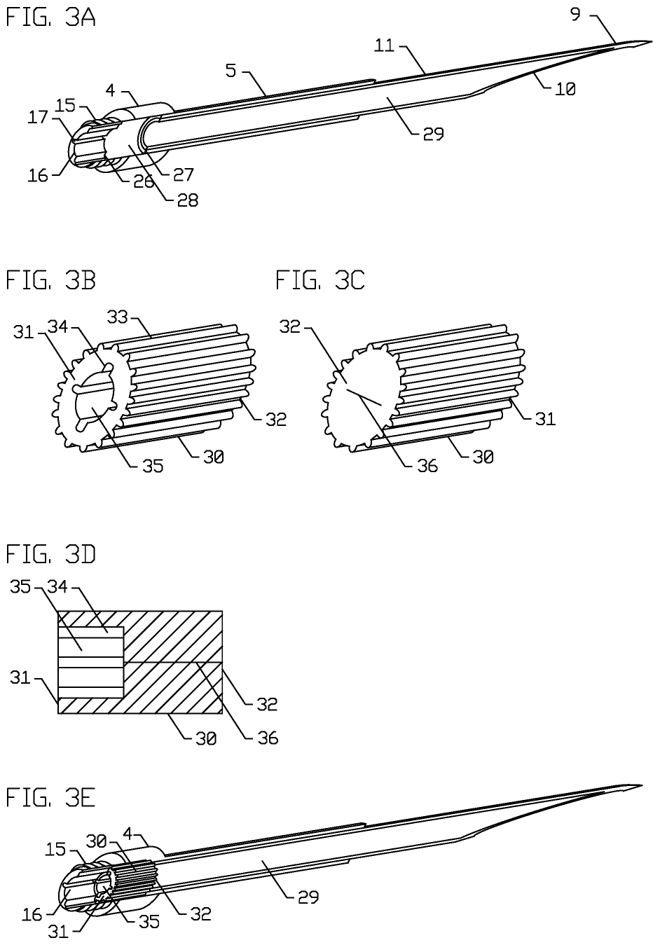

FIG. 3A depicts a schematic three-dimensional cut-out view of a portion of the outer single-lumen catheter, showing the tubular shaft 11 having a tubular space 29 and proximally opening to the elliptical proximal opening 10 which ends at the proximal tip 9 and distally adjoining the cylindrical seal chamber 4 of the sealable lock assembly of the distal portion. At the mid portion, the tubular shaft 11 is fixedly encased by the outer overtube 5. The cylindrical seal chamber 4 comprises a tubular chamber space 28 bordered proximally by a distal end 27 of the tubular shaft 11 and the outer overtube 5, and distally by a proximal end 26 of the external helical thread portion 15. The tubular chamber space 28 is coaxially open distally to the cylindrical tube 16 having the longitudinal grooves 17 of the external helical thread portion 15 and proximally to the tubular space 29 of the tubular shaft 11. FIG. 3B illustrates a schematic example of an two-part elastomeric seal 30, comprising an distal end 31 of a distal part having a cylindrical tube 35 and a proximal end 32 of the two-part elastomeric seal 30. The cylindrical tube 35 of the distal part of the two-part elastomeric seal 30 comprises a plurality of longitudinal grooves 34 disposed on an inner surface of the cylindrical tube 35. On an outer surface, the two-part elastomeric seal 30 comprises a plurality of linear ridges 33. In between a pair of the ridges, there is provided a linear furrow separating the pair of the ridges. FIG. 3C illustrates a schematic example of the two-part elastomeric seal 30, showing the proximal end 32 of a proximal part and the distal end 31. A dilatable linear slit 36 disposed longitudinally in the distal part opens to the proximal end 32. In a two dimensional view of FIG. 3D, the two-part elastomeric seal 30 comprises the cylindrical tube 35 having a plurality of the longitudinal grooves 34 open to the distal end 31, and the dilatable linear slit 36 open to the proximal end 32. FIG. 3E shows an in-situ placement of the two-part elastomeric seal 30 inside the cylindrical seal chamber 4 of the sealable lock assembly of the outer single-lumen catheter, wherein the cylindrical tube 35 open to the distal end 31 distally adjoins the cylindrical tube 16 of the external helical thread portion 15. The proximal end 32 proximally adjoins the tubular space 29 of the tubular shaft 11.

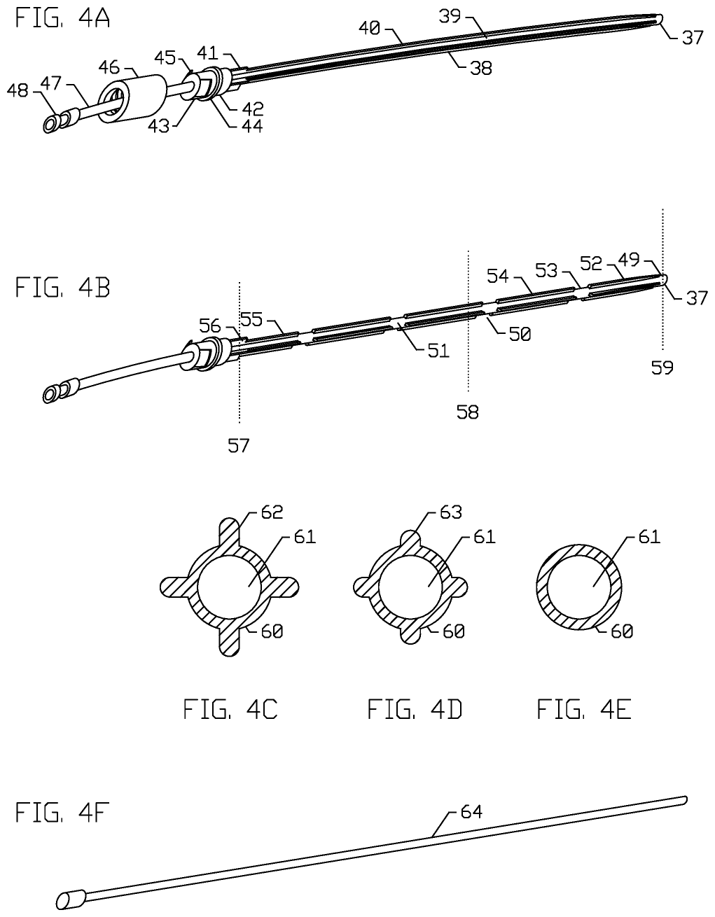

FIG. 4A shows a schematic example of components of the inner single-lumen catheter, comprising a tubular shaft 38 having a proximal end 37 provided in a circular configuration. The tubular shaft 38 distally adjoins a proximal portion 42 of a cylindrical tube of a sealable key assembly. A plurality of longitudinal ridges 40 coaxially protrude for a height from an outer surface 39 of the tubular shaft 38 along a longitudinal axis of the tubular shaft 38. Each longitudinal ridge 40 distally adjoins a rectangularly protruding tongue 41. A plurality of the rectangularly protruding tongues 41 coaxially protrude for a height from the outer surface 39 of a distal portion of the tubular shaft 38 along the longitudinal axis of the tubular shaft 38. The height of the rectangularly protruding tongue 41 is higher than the height of the longitudinal ridge 40. The rectangularly protruding tongues 41 distally adjoin the proximal portion 42 of the cylindrical tube of the sealable key assembly. The cylindrical tube of the sealable key assembly comprises the proximal portion 42, a distal portion 43, a mid circular flange 44 dividing the cylindrical tube into the proximal portion 42 and the distal portion 43. The distal portion 43 of the cylindrical tube of the sealable key assembly comprises a couple of planar leaves 45 fixedly adjoining at an angle a portion of a circumference of the distal portion 43. The distal portion 43 distally adjoins a connecting tube 47 and a connecting hub 48 that is configured to be reversibly connected to an extension tube (not shown) of a hemodialysis machine. The connecting tube 47 is configured to serve as a conduit for an outflow of blood from the hemodialysis machine to the tubular shaft 38 of the inner single-lumen catheter. A rotatable fastener 46 having an internal helical thread is slidably placed over the connecting tube 47. Referring to FIG. 2A, the rotatable fastener 46 with the internal helical thread matching with the external helical thread of the external helical thread portion 15 is configured to fasten the cylindrical tube 42˜45 of the inner single-lumen catheter to the external helical thread portion 15 of the sealable lock assembly of the outer single-lumen catheter. The planar leaves 45 are configured to distally protrude out from and abut a distal end of the rotatable fastener 46 when the rotatable fastener 46 fully fastens the cylindrical tube of the sealable key assembly of the inner single-lumen catheter to the sealable lock assembly of the outer single-lumen catheter.

FIG. 4B shows an example of discontinuous longitudinal ridges protruding from an outer surface 51 of a tubular shaft 50 of the inner single-lumen catheter. A discontinuous space is provided in between two tandem discontinuous longitudinal ridges, wherein the discontinuous space is configured to serve as flexing portion of the tubular shaft. FIG. 4B shows an example of the discontinuous space 53 in between a proximal discontinuous longitudinal ridge 52 and a distal discontinuous longitudinal ridge 54 of the tubular shaft 51. Referring to FIG. 3B, a configuration of no longitudinal ridge disposed thereof at a proximal portion 49 of the tubular shaft 50 close to the proximal end 37 is to reduce friction of the proximal portion 49 of the tubular shaft 50 with the cylindrical tube 35 of the two-part elastomeric seal 30 when the proximal portion 49 of the tubular shaft 50 enters the cylindrical tube 35. The example shown in FIG. 4B depicts a longitudinal ridge 55 disposed thereof at a distal portion of the tubular shaft 50, wherein the longitudinal ridge 55 distally adjoins a rectangularly protruding tongue 56. FIG. 4C illustrates a schematic cross-sectional view of the distal portion 57 of the tubular shaft, showing a cross-sectional view 62 of the rectangularly protruding tongue 56 protruding from a cross-sectional tubular wall 60. The cross-sectional tubular wall 60 encircles a cross-sectional tubular space 61. In FIG. 4D, a cross-sectional view of a mid portion 58 of the tubular shaft 50 shows a cross-sectional view 63 of the longitudinal ridge protruding from the cross-sectional tubular wall 60 encircling the cross-sectional tubular space 61. In FIG. 4E, a cross-sectional view of the proximal portion 59 of the tubular shaft 50 shows the cross-sectional tubular wall 60 encircling the cross-sectional tubular space 61, without the longitudinal ridges. FIG. 4F shows a schematic example of a stylet 64 that is configured to be reversibly inserted in the tubular space 60 of the inner single-lumen catheter.

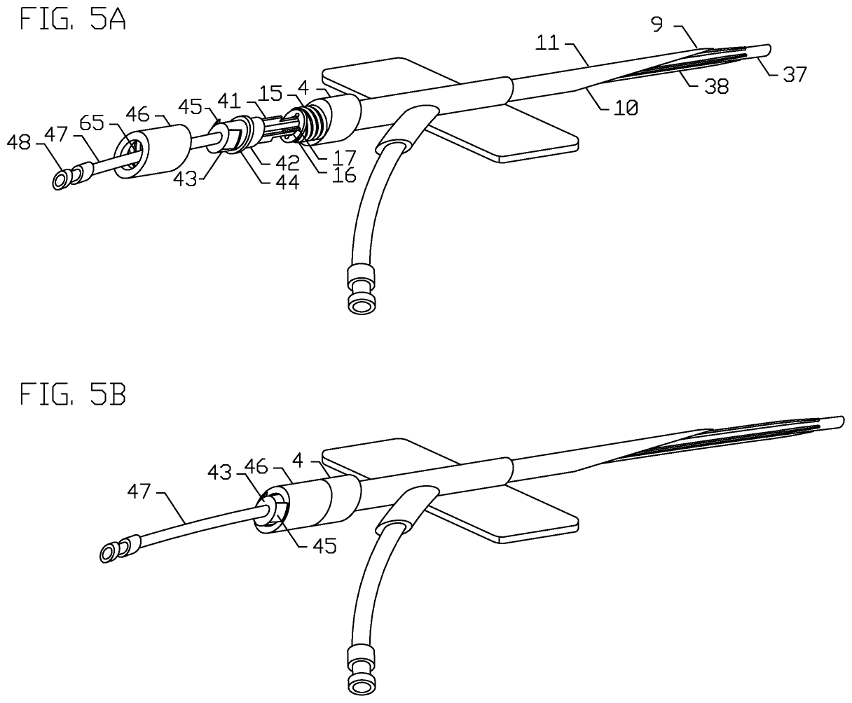

FIG. 5A shows a schematic illustration of reversible insertion of the inner single-lumen catheter having the sealable key assembly into the outer single-lumen catheter having the sealable lock assembly. The proximal end 37 of the tubular shaft 38 of the inner single-lumen catheter proximally protrudes from the elliptical proximal opening 10 of the tubular shaft 11 of the outer single-lumen catheter. In particular, the proximal end 37 of the inner single-lumen catheter proximally protrudes from the proximal tip 9 of the outer single-lumen catheter. The rectangularly protruding tongues 41 distally adjoining the proximal portion 42 of the cylindrical tube of the sealable key assembly is positioned distal to and is to insertably mate with the longitudinal grooves 17 disposed on the inner surface of the cylindrical tube 16 of the external helical thread portion 15 of the sealable lock assembly. The rotatable fastener 46 having an internal helical thread 65 is configured to rotatably slide up in a proximal direction and fasten the distal portion 43 and the proximal portion 42 of the cylindrical tube of the sealable key assembly to the external helical thread portion 15 of the sealable lock assembly. The rotatable fastener 46 is configured to slide over the planar leaves 45 and the circular flange 44 of the cylindrical tube. The distal portion 43 of the cylindrical tube of the inner single-lumen catheter is connected to the connecting tube 47 and the connecting hub 48. FIG. 5B shows a complete assembly of the sealable key assembly of the inner single-lumen catheter with the sealable lock assembly of the outer single-lumen catheter, wherein the rotatable fastener 46 tightly fastens up to the distal end of the cylindrical seal chamber 4. The planar leaves 45 projecting from the distal end of the distal portion 43 of the cylindrical tube abuts a distal end of the rotatable fastener 46, so as to prevent the rotatable fastener 46 from rotatably sliding backward over the connecting tube 47.

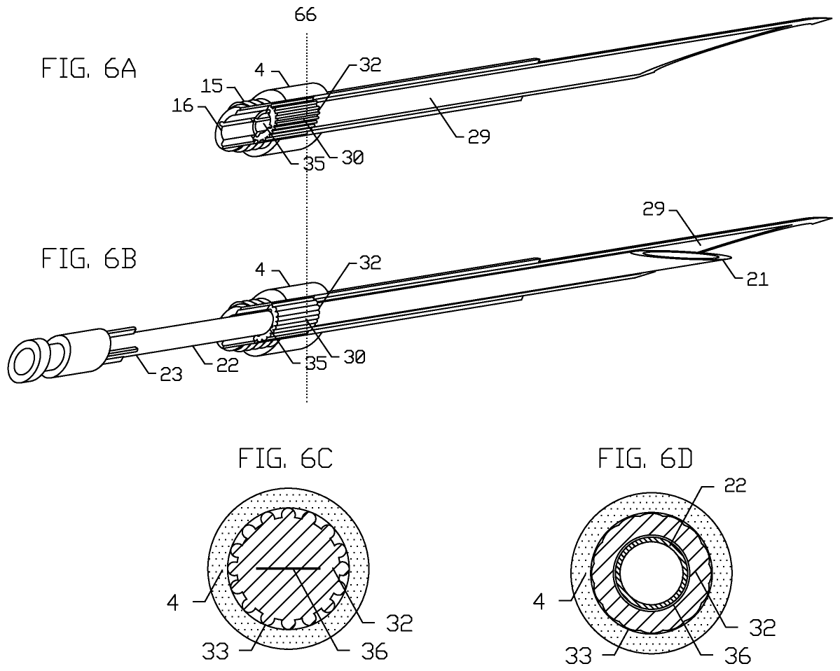

FIG. 6A shows a schematic three-dimensional cut-out view of the two-part elastomeric seal 30 encased in the cylindrical seal chamber 4 of the outer single-lumen catheter. The cylindrical tube 35 of the distal portion of the two-part elastomeric seal 30 abuts the proximal end of the external helical thread portion 15 and coaxially opens to the cylindrical tube 16 of the external helical thread portion 15. The proximal end 32 of the two-part elastomeric seal 30 proximally faces the tubular space 29 of the outer single-lumen catheter. Shown as a cross-sectional view at a proximal level 66 in FIG. 6C, the dilatable linear slit 36 of the two-part elastomeric seal 30 through the proximal end 32 of the two-part elastomeric seal 30 is closed in an unengaged configuration without the tubular needle. The linear ridges 33 of the two-part elastomeric seal 30 abutting an inner surface of the cylindrical seal chamber 4 are not centrifugally compressed in the unengaged configuration without the tubular needle. FIG. 6B shows a schematic three-dimensional cut-out view of the two-part elastomeric seal 30 in an engaged configuration with the tubular shaft portion 22 of the tubular needle having the proximal portion 21 and the distal portion 23. The tubular shaft portion 22 is penetrably inserted through the cylindrical tube 35 and the proximal end 32 of the two-part elastomeric seal 30. Shown in FIG. 6D, the dilatable linear slit 36 is centrifugally pushed open in a circular configuration by the tubular shaft portion 22 of the tubular needle while the linear ridges 33 are centrifugally compressed against the inner surface of the cylindrical seal chamber 4. The centrifugal opening of the dilatable linear slit 36 to the circular configuration is accommodated by the centrifugal compression of the linear ridges 33 against the inner surface of the cylindrical seal chamber 4.

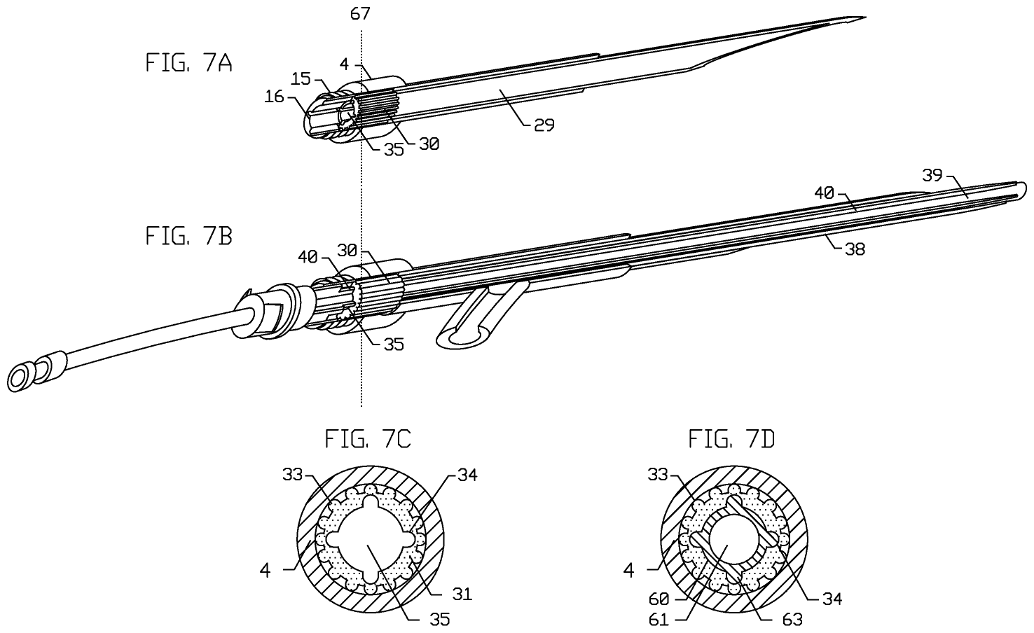

FIG. 7A shows a schematic three-dimensional cut-out view of the two-part elastomeric seal 30 encased in the cylindrical seal chamber 4 of the outer single-lumen catheter having the tubular space 29. The cylindrical tube 35 of the distal portion of the two-part elastomeric seal 30 abuts the proximal end of the external helical thread portion 15 and coaxially opens to the cylindrical tube 16 of the external helical thread portion 15. Shown as a cross-sectional view at a distal level 67 in FIG. 7C, the cylindrical tube 35 of the distal end 31 of the two-part elastomeric seal 30 having a plurality of the longitudinal grooves 34 disposed on the inner surface of the cylindrical tube 35 is open in an unengaged configuration without the sealable key assembly of the inner single-lumen catheter. The linear ridges 33 of the two-part elastomeric seal 30 abutting the inner surface of the cylindrical seal chamber 4 are not centrifugally compressed in the unengaged configuration without the sealable key assembly of the inner single-lumen catheter. FIG. 7B shows a schematic three-dimensional cut-out view of the cylindrical tube 35 of the two-part elastomeric seal 30, wherein the cylindrical tube 35 is engaged with the longitudinal ridges 40 and the outer surface 39 of the tubular shaft 38 which is penetrably inserted in the cylindrical tube 35. Shown in FIG. 7D, the tubular shaft of the inner single-lumen catheter having a cross-sectional tubular wall 60 and the longitudinal ridges 63 viewed in a cross-section of the longitudinal ridges 40 is seen as sealably mating with the cylindrical tube 35 and the longitudinal grooves 34. A cross-sectional tubular space of the inner single-lumen catheter is shown as 61. The linear ridges 33 remain unchanged, i.e., not centrifugally compressed against the inner surface of the cylindrical seal chamber 4, in the engaged configuration with the tubular shaft of the inner single-lumen catheter having the cross-sectional tubular wall 60 and the longitudinal ridges 63.

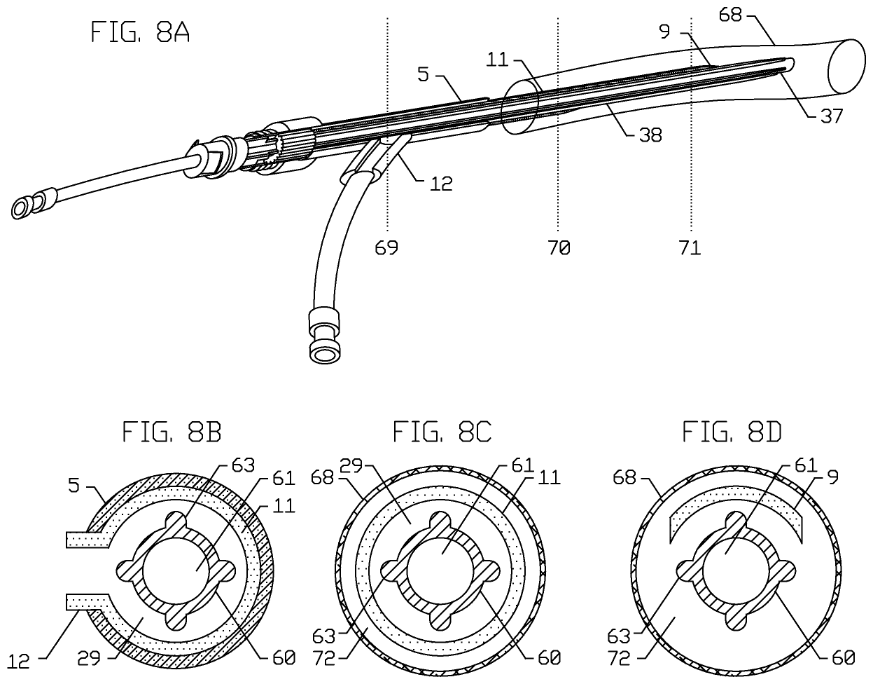

FIG. 8A shows a schematic three-dimensional cut-out and see-through view of an example of the arteriovenous fistula access device inserted inside a blood vessel 68. The arteriovenous fistula access device comprises the inner single-lumen catheter inserted in the outer single-lumen catheter. A proximal portion of the arteriovenous fistula access device is inserted in the blood vessel 68, wherein the proximal portion of the arteriovenous fistula access device comprises the tubular shaft 38 and the proximal end 37 of the inner single-lumen catheter protruding from the tubular shaft 11 and the proximal tip 9 of the outer single-lumen catheter. A cross-sectional schematic view of the arteriovenous fistula access device at a level 69 of the mid portion of the outer single-lumen catheter is shown in FIG. 8B, illustrating the outer overtube 5 encasing the tubular shaft 11. One sidewall of the outer overtube 5 is open through which the connecting tubular flange 12 adjoins and opens into a sidewall of the tubular shaft 11. The tubular space 29 of the outer single-lumen catheter slidably encloses the cross-sectional view 63 of the longitudinal ridges protruding from the tubular wall 60 of the tubular shaft 38 of the inner single-lumen catheter. The tubular space of the tubular shaft 38 of the inner single-lumen catheter is depicted as 61.

A cross-sectional schematic view of the arteriovenous fistula access device at a level 70 of a distal part of the proximal portion of the outer single-lumen catheter is shown in FIG. 8C, showing the tubular shaft 11 in a circular configuration inserted in the blood vessel 68 having a vascular space 72. The cross-sectional schematic view of the arteriovenous fistula access device shows compartmentalized tubular spaces comprising the tubular space 29 in between the tubular shaft 11 of the outer single-lumen catheter and the tubular wall 60 of the inner single-lumen catheter, and the tubular space 61 of the inner single-lumen catheter. The tubular space 29 is configured to bring in an inflow of blood from the vascular space 72, whereas the tubular space 61 is configured to return the blood back into the vascular space 72. The longitudinal ridges 63 of the inner single-lumen catheter are configured to help maintain the tubular space 29 against a centripetal collapse of the tubular shaft 11 toward the tubular wall 60 by a negative pressure inside the tubular space 29 generated by a hemodialysis machine. A cross-sectional schematic view of the arteriovenous fistula access device at a level 71 near the proximal tip 9 of the outer single-lumen catheter is shown in FIG. 8D, showing the proximal tip 9 inside the vascular space 72 of the blood vessel 68. The longitudinal ridges 63 are configured to prevent a centripetal collapse of the proximal tip 9 toward the tubular wall 60 of the inner single-lumen catheter by the negative pressure inside the tubular space 29 generated by the hemodialysis machine. At this level 71, the tubular space 61 of the inner single-lumen catheter is maintained intact.

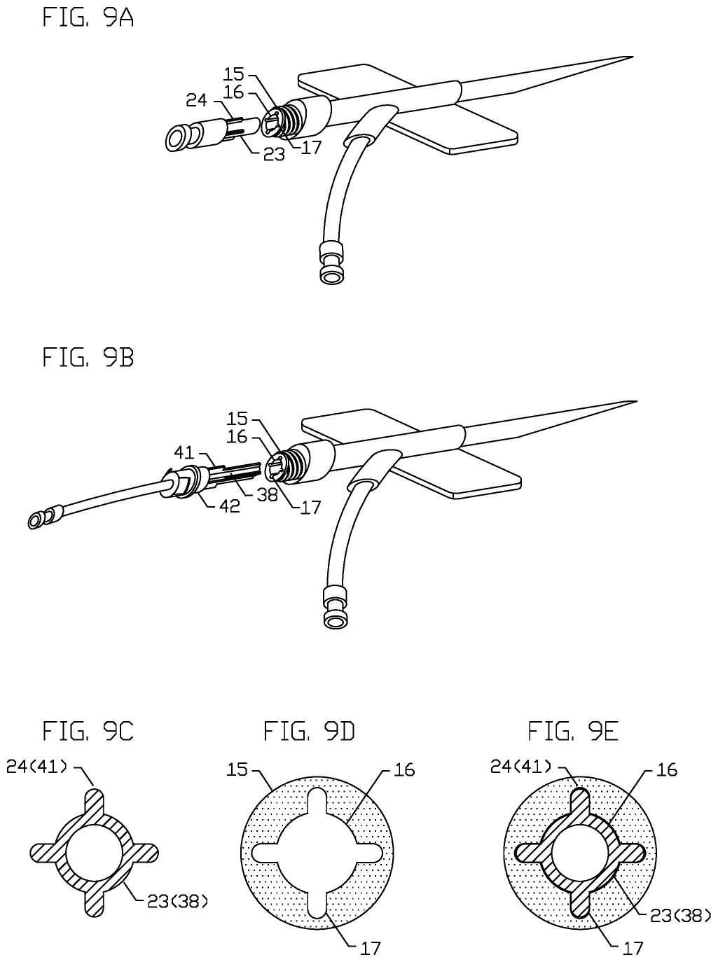

FIG. 9A shows a schematic view of the external helical thread portion 15 of the sealable lock assembly of the outer single-lumen catheter and the distal portion 23 of the tubular needle having a plurality of the rectangularly protruding tongues 24 circumferentially disposed thereof at the distal portion of the tubular needle. The rectangularly protruding tongues 24 are configured to slidably mate with the longitudinal grooves 17 of the cylindrical tube 16 of the external helical thread portion 15. The distal portion 23 of the tubular needle is configured to slidably mate with the cylindrical tube 16 of the external helical thread portion 15. In FIG. 9B, the cylindrical tube 16 of the external helical thread portion 15 is configured to mate with the distal portion of the tubular shaft 38 of the inner single-lumen catheter. The longitudinal grooves 17 of the cylindrical tube 16 is configured to slidably mate with the rectangularly protruding tongues 41 proximally projecting from the proximal cylindrical tube 42 of the sealable key assembly of the inner single-lumen catheter. A cross-sectional configuration of the distal portion 23 and the rectangularly protruding tongues 24 of the tubular needle is the same as that of the distal portion and the rectangularly protruding tongues 41 of the tubular shaft 38. FIG. 9C shows an identical schematic cross-sectional configuration of the distal portion 23 and the rectangularly protruding tongues 24 of the tubular needle to the distal portion of the tubular shaft (38) and the rectangularly protruding tongues (41) of the tubular shaft 38, respectively. FIG. 9D shows a schematic cross-sectional view of the external helical thread portion 15 having the cylindrical tube 16 and a plurality of the longitudinal grooves 17. FIG. 9E shows a fully mated configuration of the cylindrical tube 16 and a plurality of the longitudinal grooves 17, with the distal portion 23 and the rectangularly protruding tongues 24 of the tubular needle, and the distal portion of the tubular shaft (38) and the rectangularly protruding tongues (41) of the tubular shaft 38 of the inner single-lumen catheter, respectively.

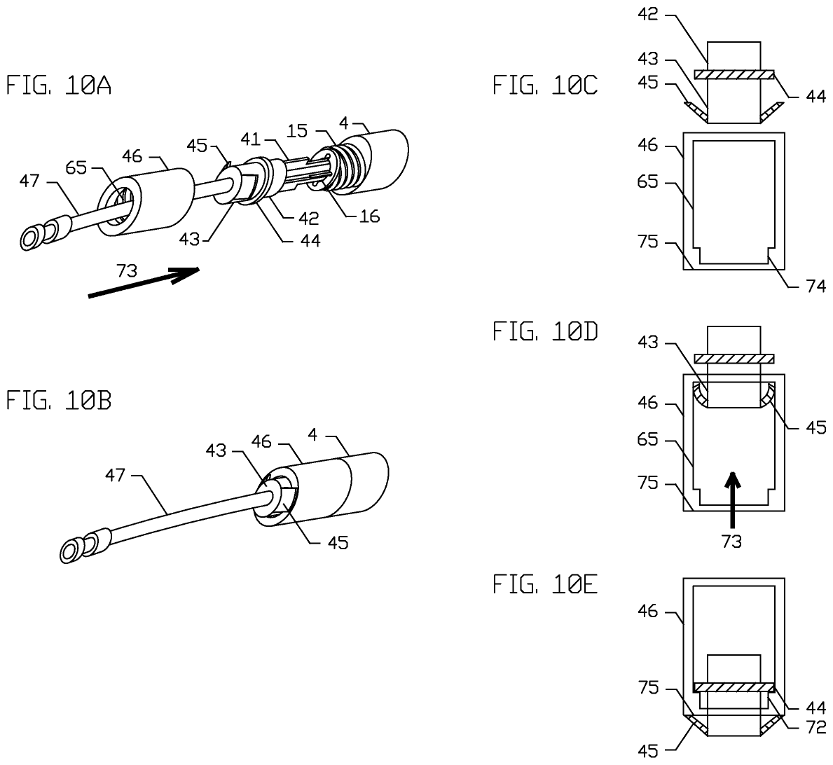

FIG. 10A shows a schematic view of the rotatable fastener 46 of the inner single-lumen catheter fastening the sealable key assembly of the inner single-lumen catheter to the sealable lock assembly of the outer single-lumen catheter. The sealable key assembly comprises a plurality of the rectangularly protruding tongues 41, the cylindrical tube divided by the circular flange 44 into the proximal portion 42 and distal portion 43, and a couple of the planar leaves 45. The sealable key assembly is coaxially arranged with the sealable lock assembly of the outer single-lumen catheter which comprises the cylindrical seal chamber 4, and the external helical thread portion 15 having the cylindrical tube 16 inside the external helical thread portion 15. The rotatable fastener 46 having the internal helical thread 65 slides over the connecting tube 47 so as to meet with the sealable key assembly in a proximal direction 73. Shown in FIG. 10B, the rotatable fastener 46 tightly fastens the sealable key assembly to the sealable lock assembly, wherein the proximal end of the rotatable fastener 46 abuts the distal end of the cylindrical seal chamber 4, and wherein a distal end of the rotatable fastener 46 is pushed proximally by the planar leaves 45 projecting from the distal end of the distal portion 43 of the cylindrical tube of the sealable key assembly. The planar leaves 45 are configured to prevent the rotatable fastener 46 from distally sliding out.

FIG. 10C shows a schematic two-dimensional see-through view of the rotatable fastener 46 comprising the distal end 75, a distal inner circular flange 74 and the internal helical thread 65. In a schematic two-dimensional see-through view, the sealable key assembly comprises the cylindrical tube divided by the circular flange 44 into the proximal portion 42 and distal portion 43, and a couple of the planar leaves 45. When the rotatable fastener 46 is advanced in the proximal direction 73 over the sealable key assembly, shown in FIG. 10D, the planar leaves 4 are configured to proximally bend and fall inside the internal helical thread 65 of the rotatable fastener 46. FIG. 10E shows a schematic two-dimensional see-through configuration of a full fastening of the sealable key assembly by the rotatable fastener 46, wherein the distal inner circular flange 74 of the rotatable fastener 46 abuts the circular flange 44 of the sealable key assembly, and wherein the planar leaves 45 abuts the distal end 75 of the rotatable fastener 46.

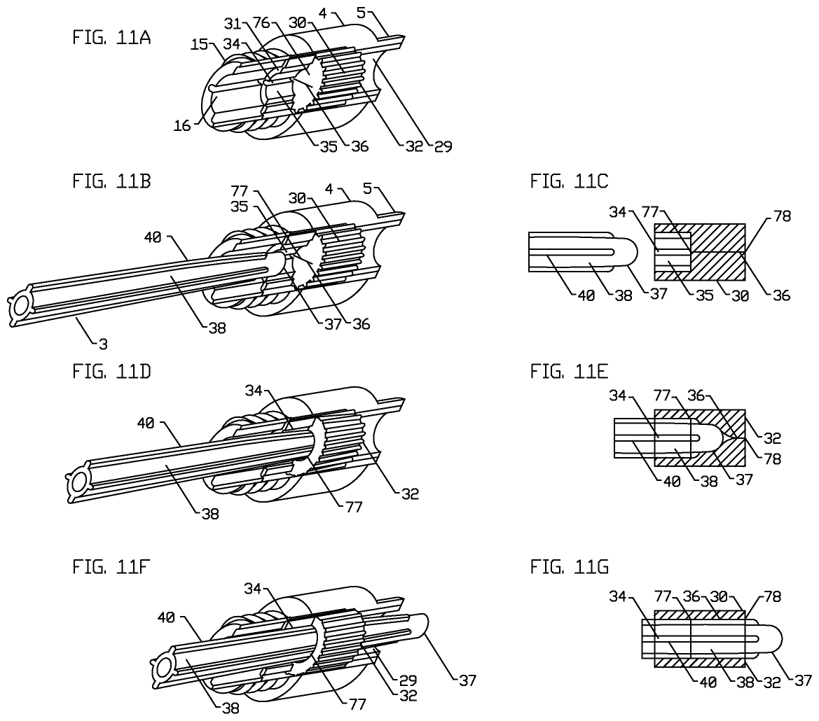

FIG. 11A shows the two-part elastomeric seal 30 immovably disposed in the cylindrical seal chamber 4 of the sealable lock assembly of the outer single-lumen catheter, proximal to the cylindrical tube 16 of the external helical thread portion 15 and distal to the tubular space 29 of the overtube 5 of the outer single-lumen catheter. The distal part of the two-part elastomeric seal 30 is exposed, showing the distal end 31 and the cylindrical tube 35 with the plurality of the longitudinal grooves 34 on the inner surface of the cylindrical tube 35. A distal face 76 of the solid elastomeric cylinder having the dilatable linear slit 36 is shown, along with the proximal end 32 of the two-part elastomeric seal 30. In FIGS. 11B and 11C, the cylindrical tube 35 of the two-part elastomeric seal 30 is about to be penetrated into by the proximal end 37 of the proximal portion 3 of the inner single-lumen catheter having the tubular shaft 38 with the plurality of the longitudinal ridges 40. A distal opening 77 of the dilatable linear slit 36 of the two-part elastomeric seal 30 is not yet open as is a proximal opening 78 of the dilatable linear slit 36.

FIGS. 11D and 11E show the proximal end 37 of the tubular shaft 38 of inner single-lumen catheter is just inserted in the distal opening 77 of the two-part elastomeric seal 30 but not through the proximal end 32 of the two-part elastomeric seal 30, illustrating the first step of seal by the proximal end 32 of the two-part elastomeric seal 30. The plurality of the linear grooves 34 are beginning to make a leakproof contact with the plurality of the linear ridges 40. The proximal end 37 thrustingly opens the distal opening 77 of the dilatable linear slit 36, yet the proximal opening 78 disposed at the proximal end 32 stays closed shut. FIGS. 11F and 11G show a full penetration of the tubular shaft 38 of the proximal portion of the inner single-lumen catheter through the two-part elastomeric seal 30 into the tubular space 29. The plurality of the linear grooves 34 are leakproofly mating with the plurality of the linear ridges 40, the distal opening 77 is fully opened by the tubular shaft 38, and the proximal opening 78 disposed at the proximal end 32 is now dilatably opened by the proximal end 37. The plurality of the linear grooves 34 leakproofly mating with the plurality of the linear ridges 40 accomplish the second step of the seal of the two-part elastomeric seal 30 during the introduction of the inner single-lumen catheter in the outer single-lumen catheter.

It is to be understood that the aforementioned description of the vascular access apparatus is simple illustrative embodiments of the principles of the present invention. Various modifications and variations of the description of the present invention are expected to occur to those skilled in the art without departing from the spirit and scope of the present invention. Therefore the present invention is to be defined not by the aforementioned description but instead by the spirit and scope of the following claims.

What is claimed is:

1. A vascular access apparatus for vascular access, comprising:
an outer single-lumen catheter, and an inner single-lumen catheter comprising a plurality of longitudinal ridges disposed on an outer surface of a tubular shaft of the inner single-lumen catheter, wherein the inner single-lumen catheter is configured to be reversibly threaded in a tubular space of the outer single-lumen catheter so as to reversibly compartmentalize a single lumen of the outer single-lumen catheter into a coaxial double lumen.

2. The vascular access apparatus for vascular access according to claim 1, wherein the outer single-lumen catheter further comprises:
a proximal opening, wherein the proximal opening is provided in a beveled, elliptical configuration having a long radius of the proximal opening longer than 2×a short radius of the proximal opening so as to reduce turbulence of an inflow of blood flowing through the proximal opening and cross-sectional collapse of the proximal opening by the inflow of the blood.

3. The vascular access apparatus for vascular access according to claim 1, wherein the outer single-lumen catheter further comprises:
a distal portion having a sealable lock assembly;
the sealable lock assembly comprises an external helical thread portion adjoining proximally a seal portion, wherein the sealable lock assembly is configured to sealably accommodate and lock the inner single-lumen catheter in the outer single-lumen catheter.

4. The vascular access apparatus for vascular access according to claim 3, wherein the sealable lock assembly further comprises:
the external helical thread portion, provided with an external helical thread disposed on an outer surface of a cylindrical tube of the external helical thread portion, and a plurality of longitudinal grooves radially disposed on an inner surface of the cylindrical tube; and
the seal portion of the sealable lock assembly, comprising a cylindrical seal chamber sealably encasing a two-part elastomeric seal, wherein a distal part of the two-part elastomeric seal is provided in a cylindrical tubular configuration and non-dilatable, and wherein a proximal part of the two-part elastomeric seal comprises a solid elastomeric cylinder having a dilatable linear slit provided coaxially in said solid elastomer cylinder.

5. The vascular access apparatus for vascular access according to claim 4, wherein the two-part elastomeric seal further comprises:
a plurality of longitudinal ridges and furrows axially disposed on an outer circumferential surface of the two-part elastomeric seal in a configuration of a corrugated roll;
wherein the plurality of the longitudinal ridges of the two-part elastomeric seal radially protruding from the outer circumferential surface in an unengaged configuration is configured to seal the cylindrical seal chamber; and
wherein the plurality of the longitudinal ridges of the two-part elastomeric seal are configured to be compressible against an inner wall of the cylindrical seal chamber in an engaged configuration with the inner single-lumen catheter penetrably introduced inside the dilatable linear slit of the two-part elastomeric seal.

6. The vascular access apparatus for vascular access according to claim 4, wherein the two-part elastomeric seal further comprises:
a radius of the dilatable linear slit in a dilated configuration engaged with the inner single-lumen catheter is configured to be equal to or less than a square root of a sum of [a radius² of the two-part elastomeric seal from an axial center to an outer periphery of the plurality of the longitudinal ridges of the two-part elastomeric seal in a uncompressed configuration minus a radius² of the two-part elastomeric seal from the axial center to the outer periphery of the plurality of the longitudinal ridge of the two-part elastomeric seal in a compressed configuration].

7. The vascular access apparatus for vascular access according to claim 4, wherein the two-part elastomeric seal further comprises:
a plurality of longitudinal grooves axially disposed on an inner circumferential surface of the distal part of the two-part elastomeric seal;
wherein the plurality of the longitudinal grooves of the distal part of the two-part elastomeric seal are configured to be leakproofly engaged with the plurality of the longitudinal ridges of the inner single-lumen catheter.

8. The vascular access apparatus for vascular access according to claim 4, wherein the two-part elastomeric seal further comprises:
the dilatable linear slit of the proximal part is configured to stay closed until a proximal portion of the plurality of the longitudinal ridges of the inner single-lumen catheter leakproofly mate with the plurality of the longitudinal grooves of the distal part of the two-part elastomeric seal.

9. The vascular access apparatus for vascular access according to claim 1, wherein the inner single-lumen catheter further comprises:
the plurality of the longitudinal ridges comprising each longitudinal ridge radially arising from the outer surface of the tubular shaft of the inner single lumen catheter;
wherein the longitudinal ridge is configured to separate an inner surface of the outer single-lumen catheter from the outer surface of the tubular shaft of the inner single-lumen catheter by a vertical height of the longitudinal ridge in a configuration of the tubular shaft of the the inner single-lumen catheter coaxially threaded in the tubular space of the outer single-lumen catheter;
wherein separation of the inner surface of the outer single-lumen catheter from the outer surface of the tubular shaft of the inner single-lumen catheter produces a longitudinal tubular space; and
wherein the longitudinal tubular space is configured to serve as a conduit for the inflow of the blood whereas the inner single-lumen catheter serves a conduit for an outflow of the blood.

10. The vascular access apparatus for vascular access according to claim 1, wherein the inner single-lumen catheter further comprises:
a proximal portion having a proximal opening in a circular configuration;
wherein the proximal portion of the inner single-lumen catheter is configured to proximally protrude for a length from the proximal opening of the outer single-lumen catheter in an engaged configuration with said outer single-lumen catheter so as to avoid recirculation of blood between the proximal opening of the outer single-lumen catheter and the proximal opening of the inner single-lumen catheter.

11. The vascular access apparatus for vascular access according to claim 1, wherein the inner single-lumen catheter further comprises:
a distal portion having a sealable key assembly, and a rotatable fastener slidably placed over a connecting tube;
the sealable key assembly, provided as a cylindrical tube proximally connected to the tubular shaft of the inner single-lumen catheter and distally adjoining the connecting tube of the inner single-lumen catheter;
wherein the sealable key assembly is configured with a plurality of rectangularly protruding tongues circumferentially disposed thereof on an outer surface of the sealable key assembly; and
wherein the plurality of the rectangularly protruding tongues are configured to sealably mate with the plurality of the longitudinal grooves of the sealable lock assembly of the outer single-lumen catheter; and
the rotatable fastener, provided with a matching internal helical thread to the external helical thread of the sealable lock assembly of the outer single-lumen catheter, wherein the rotatable fastener is configured to reversibly fasten the sealable key assembly to the sealable lock assembly of the outer single-lumen catheter.

12. The vascular access apparatus for vascular access according to claim 5, wherein the inner single-lumen catheter further comprises:
a couple of planar leaves of the sealable key assembly fixedly adjoining at an angle a distal end of the cylindrical tube of the sealable key assembly;
wherein a planar leaf is provided as a thermoplastic rectangular plate; and
wherein the couple of the planar leaves are configured to distally protrude out from and abut a distal end of the rotatable fastener so as to prevent backward slippage of the inner single-lumen catheter from the outer single-lumen catheter.
